(19) United States
(12) Patent Application Publication
(10) Pub. No.: US 2026/0213021 A1
(43) Pub. Date: Jul. 23, 2026

(54) RISK ESTIMATION DEVICE, RISK ESTIMATION METHOD, AND RECORDING MEDIUM

(71) Applicant: NEC Corporation, Tokyo (JP)

(72) Inventors: Chenhui HUANG, Tokyo (JP); Kensuke Wagata, Tokyo (JP); Fumiyuki Nihey, Tokyo (JP)

(73) Assignee: NEC Corporation, Tokyo (JP)

(21) Appl. No.: 19/452,939

(22) Filed: Jan. 20, 2026

(30) Foreign Application Priority Data
Jan. 23, 2025 (JP) 2005-009875

(51) Int. Cl. G16H 50/30; G06N 20/00; G16H 10/60

(52) U.S. Cl. CPC G16H 50/30; G06N 20/00; G16H 10/60

(57) ABSTRACT

In the risk estimation device, the conversion means converts input data into a feature. The first processing means converts the feature into a latent variable indicating a probability distribution in the latent space. The query generation means generates query data. The second processing means performs attention processing on the query data and the latent variable using the attention mechanism, and outputs the latent variable after the attention processing. The estimation means estimates a risk based on the latent variable after the attention processing. By using a machine learning model trained by the training device to estimate disease risk, it is possible to support decision-making regarding the lifestyle habits of subjects.

### INCORPORATION BY REFERENCE

This application is based upon and claims the benefit of priority from Japanese Patent Application 2025-009875, filed on Jan. 23, 2025, the disclosure of which is incorporated herein in its entirety by reference.

### TECHNICAL FIELD

The present disclosure relates to risk estimation.

### BACKGROUND ART

A disease risk estimation technique using a machine learning model is known. For example, WO2023/276976 describes a machine learning model that predicts progression of dementia using multiple types of input data. In WO2023/276976, a final prediction result is generated by integrating prediction results based on multiple pieces of input data, according to a prediction interval from a reference time point to a future time point being predicted.

### SUMMARY

In the method of WO2023/276976, since the prediction results based on the multiple pieces of input data are integrated according to the prediction interval, a highly accurate prediction result is not necessarily obtained.

One object of the present disclosure is to provide a risk estimation device capable of highly accurate risk estimation.

According to an example aspect of the present invention, there is provided a risk estimation device comprising:
a conversion means configured to convert input data into a feature;
a first processing means configured to convert the feature into a latent variable indicating a probability distribution in a latent space;
a query generation means configured to generate query data;
a second processing means configured to perform attention processing on the query data and the latent variable using an attention mechanism, and output the latent variable after the attention processing; and
an estimation means configured to estimate a risk based on the latent variable after the attention processing.

According to another example aspect of the present invention, there is provided a risk estimation method executed by a computer, the risk estimation method comprising:
converting input data into a feature;
converting the feature into a latent variable indicating a probability distribution in a latent space;
generating query data;
performing attention processing on the query data and the latent variable using an attention mechanism and outputting the latent variable subjected to the attention processing; and
estimating a risk based on the latent variable subjected to the attention processing.

According to still another example aspect of the present invention, there is provided a program causing a computer to execute processing comprising:
converting input data into a feature;
converting the feature into a latent variable indicating a probability distribution in a latent space;
generating query data;
performing attention processing on the query data and the latent variable using an attention mechanism and outputting the latent variable subjected to the attention processing; and
estimating a risk based on the latent variable subjected to the attention processing.

### Effect

According to the present disclosure, highly accurate risk estimation can be achieved.

### BRIEF DESCRIPTION OF THE DRAWINGS

FIG. 1 illustrates an overall configuration of a risk estimation device according to the present disclosure;

FIG. 2 is a block diagram illustrating a hardware configuration of the risk estimation device;

FIG. 3 is a block diagram illustrating a functional configuration of a risk estimation model training device;

FIGS. 4A to 4D illustrate a configuration example of each unit of the training device;

FIG. 5 is a flowchart of training processing;

FIG. 6 is a block diagram illustrating a functional configuration of the risk estimation device;

FIG. 7 is a flowchart of the risk estimation processing;

FIG. 8 is a block diagram illustrating another configuration example of the risk estimation device;

FIGS. 9A and 9B illustrate a comparison of an estimation result and performance of the risk estimation device;

FIG. 10 is a block diagram illustrating another configuration example of the risk estimation device;

FIG. 11 is a block diagram illustrating another configuration example of the risk estimation device;

FIG. 12 is a block diagram illustrating another configuration example of the risk estimation device;

FIG. 13 is a block diagram illustrating another configuration example of the risk estimation device;

FIG. 14 is a block diagram illustrating another configuration example of the risk estimation device;

FIG. 15 illustrates another configuration example of an attention module;

FIG. 16 is a block diagram illustrating a configuration of another risk estimation device of the present disclosure; and

FIG. 17 is a flowchart of another risk estimation processing.

### EXAMPLE EMBODIMENTS

Preferred example embodiments of the present disclosure will be described with reference to the accompanying drawings.

### DESCRIPTION OF BACKGROUND

When some prediction or estimation is performed based on a large amount of input information, it is required to efficiently compress the input information. One method of information compression is the information bottleneck (hereinafter referred to as the “IB”). The IB is a framework based on information theory, and provides a method for efficiently extracting and compressing related information. Specifically, the IB considers the problem of “Encode information on another variable Y into as small space as possible while keeping this information as much as possible” based on the information theory. The IB can also achieve removal of redundant information and noise. The concept of the IB is described in Document 1 below, the contents of which are incorporated herein by reference.

### (Document 1)

Alemi, A. A., Fischer, I., Dillon, J. V., & Murphy, K. (2016). Deep variational information bottleneck. arXiv preprint arXiv: 1612.00410.

Another method of information compression is the transformer using cross-attention. Hereinafter, this is also referred to as the “perceiver”. Although the perceiver is not based on the information theory, data-driven information compression is practically achieved by forming a bottleneck by the input structure of an attention mechanism. The perceiver is described in Document 2 below, the contents of which are incorporated herein by reference.

### (Document 2)

Andrew Jaegle, Felix Gimeno, Andrew Brockm, Andrew Zisserman, Oriol Vinyals, Joao Carreira, Perceiver: General Perception with Iterative Attention, arXiv: 2103.03206.

The IB is intended to hold important information and remove unnecessary noise. However, if the information is excessively reduced, necessary information is also lost, causing a risk that the correlation between the bottleneck and the original data is lost. On the other hand, although the perceiver supports huge amount of data, selection for appropriately extracting important information is complicated. Also, since it is completely data-driven, there is a risk of overfitting to training data.

Thus, the present disclosure fuses both the IB and the perceiver and reinforces them with each other. As a result, it can be expected to complement each other's weak points with three aspects of efficient extraction and compression of information, reduction in calculation cost, and prevention of overfitting, thereby to improve the overall performance. In addition, it is possible to efficiently and effectively perform data processing and training by synergistically utilizing the strengths of the respective methods.

### First Example Embodiment

### [Overall Configuration]

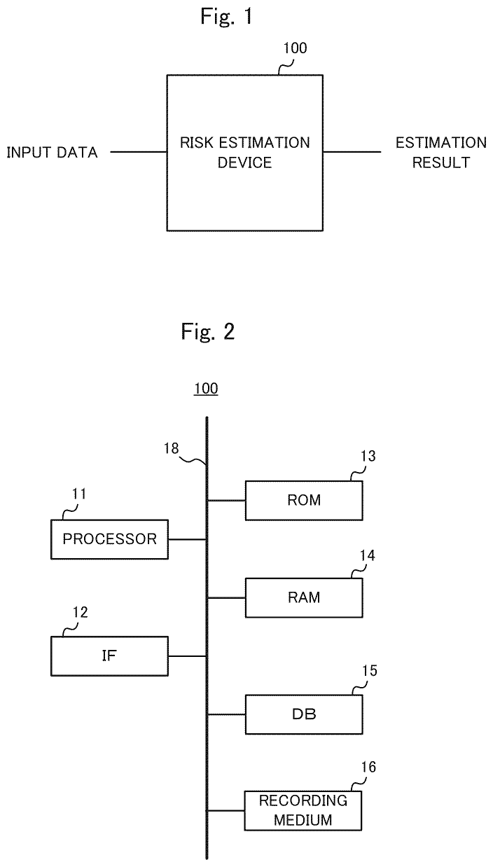

FIG. 1 illustrates an overall configuration of a risk estimation device according to the present disclosure. The risk estimation device 100 estimates a disease risk of a subject based on health data of the subject. Specifically, input data on the health of the subject is input to the risk estimation device 100. The input data includes, for example, various pieces of data obtained by medical examination or the like, such as height, weight, sex, blood pressure, body mass index (BMI), body fat percentage, neutral fat value, smoking status and amount, drinking status and amount, and the like of the subject. Note that, the input data may be multimodal data as will be described later, that is, multiple pieces of data among the above.

The risk estimation device 100 estimates the disease risk of the subject using a previously trained risk estimation model. As illustrated in FIG. 1, input data on the health of the subject is input to the risk estimation device 100. The risk estimation device 100 estimates the disease risk based on the input data and outputs an estimation result. At this time, the risk estimation device 100 compresses information by using a variational information bottleneck (VIB) and a transformer (perceiver). Note that, the VIB is a specific method obtained by extending the IB to a form that can be used in a machine training model. This enables the risk estimation device 100 to estimate the disease risk with high accuracy.

The risk estimation device 100 can be suitably applied in the medical or healthcare field. For example, the risk estimation device 100 can be used to estimate the risk of a lifestyle-related disease based on data obtained in a regular medical examination.

### [Hardware Configuration]

FIG. 2 is a block diagram illustrating a hardware configuration of the risk estimation device 100. As illustrated, the risk estimation device 100 includes a processor 11, an interface (IF) 12, a read only memory (ROM) 13, a random access memory (RAM) 14, a database (DB) 15, and a recording medium 16. Those components are connected to each other via a bus 18, for example.

The processor 11 is a computer such as a central processing unit (CPU), and controls the entire risk estimation device 100 by executing a program prepared in advance. Specifically, as the processor 11, a CPU, a graphics processing unit (GPU), a digital signal processor (DSP), a micro processing unit (MPU), a floating point number processing unit (FPU), a physics processing unit (PPU), a tensor processing unit (TPU), a quantum processor, a microcontroller, or a combination of these can be used.

In addition, the processor 11 loads a program stored in the ROM 13 or the recording medium 16 into the RAM 14 and executes each process coded in the program. The processor 11 functions as a part or all of the risk estimation device 100. Specifically, the processor 11 executes training processing and risk estimation processing to be described later.

The IF 12 transmits and receives data to and from an external device. Specifically, in a training phase, the risk estimation device 100 receives input data on the health of multiple persons as training data through the IF 12. Further, in an estimation phase, that is, at the time of risk estimation, the risk estimation device 100 receives the input data on the health of the subject through the IF 12 and outputs an estimation result of the disease risk to a display device or another external device.

The ROM 13 stores various programs to be executed by the processor 11. The RAM 14 is used as a working memory during execution of various types of processing by the processor 11.

The DB 15 stores various algorithms, data, machine learning models, and the like used when the risk estimation device 100 executes the training processing and risk estimation processing to be described later.

The recording medium 16 is a non-volatile non-transitory storage medium such as a disk-shaped recording medium or a semiconductor memory. The recording medium 16 may be configured to be detachable from the risk estimation device 100. The recording medium 16 records various programs to be executed by the processor 11.

In addition to the above, the risk estimation device 100 may include a display device such as a liquid crystal display and an input device such as a keyboard and a mouse. The display and input devices are used by an operator of the risk estimation device 100, for example.

### [Training Phase]

Next, the training phase of the risk estimation model will be described.

### (Training Device)

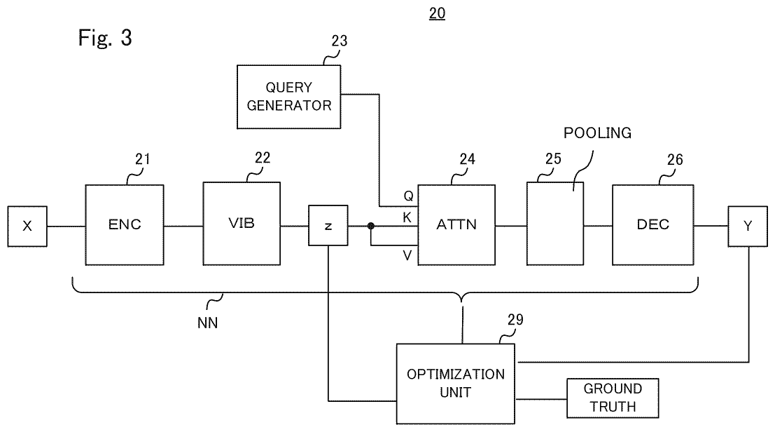

The risk estimation device 100 estimates the disease risk using a trained risk estimation model. FIG. 3 is a block diagram illustrating a functional configuration of a training device 20 of a risk estimation model. The training device 20 includes an encoder 21, a VIB 22, a query generator 23, an attention module 24, a pooling layer 25, a decoder 26, and an optimization unit 29.

The risk estimation model is configured by the encoder 21, the VIB 22, the query generator 23, the attention module 24, the pooling layer 25, and the decoder 26. Here, the encoder 21, the VIB 22, the attention module 24, the pooling layer 25, and the decoder 26 are configured by a neural network NN. In the training phase, the training device 20 generates a trained risk estimation model by optimizing this neural network NN using training data.

As the training data, multimodal disease risk data on multiple persons is prepared. Specifically, the training data is data obtained by collecting attribute data and disease risk values of the multiple persons. As the attribute data, for example, those having high relevance to the disease risk to be estimated among height, weight, sex, blood pressure, BMI, neutral fat value, blood glucose level, smoking status and amount, drinking status and amount, and the like are used. Note that, the disease risk value of each person corresponds to correct answer data in so-called supervised learning, and is hereinafter also referred to as a “ground truth”. For example, it is assumed that the risk of heart disease is estimated as the disease risk using the blood pressure, BMI, and neutral fat value as input data X. In this case, the training data includes, for each of the multiple persons, data including blood pressure, BMI, and neutral fat value as the input data and the presence or absence of heart disease as the ground truth.

In FIG. 3, the input data X related to the health of the subject is input to the encoder 21. The encoder 21 projects the input data to a latent space. The “latent space” is an abstract space for expressing information included in original data in fewer dimensions, and in the latent space, essential features and patterns of data are expressed in fewer dimensions. The expression “projects . . . to a latent space” refers to converting the original data into points on the latent space, which is also referred to as “mapping to the latent space”. Specifically, the encoder 21 converts the input data into an embedding vector that is a feature in the latent space.

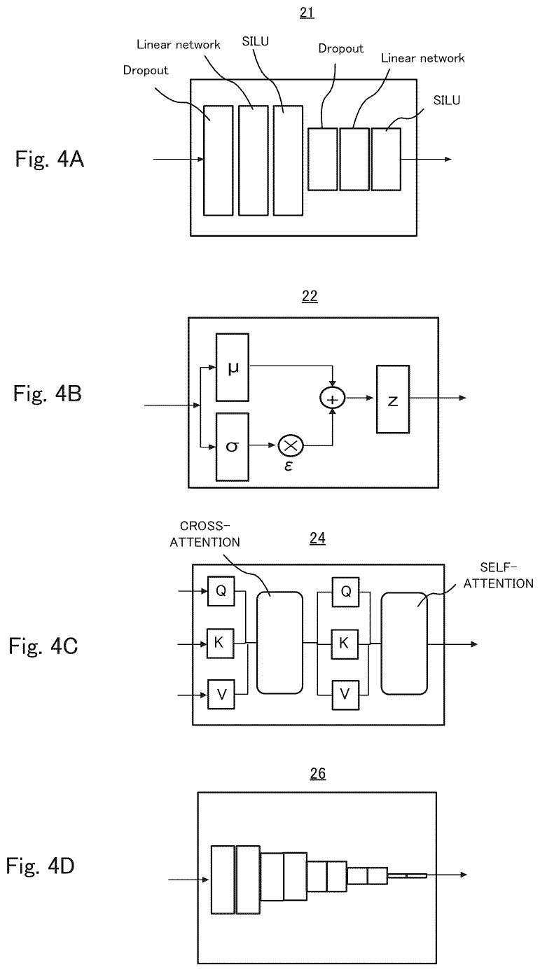

FIG. 4A illustrates a configuration example of the encoder 21. In the example of FIG. 4A, the encoder 21 is configured by connecting multiple networks including a dropout layer, a linear network, and a Sigmoid Linear Unit (SILU). The dropout layer invalidates neurons in the neural network with a certain probability at the time of training. The linear network is a network in which an activation function in each layer is configured by a linear function. The SILU is an activation function. The encoder 21 outputs a vector obtained by converting the input data to the VIB 22 as the feature.

The VIB 22 calculates a probability distribution in the latent space of the input data based on the input feature, that is, the vector in the latent space, and generates a latent variable z indicating the probability distribution. FIG. 4B illustrates a configuration example of the VIB 22. The probability distribution is indicated by a mean μ and standard deviation σ. The latent variable z is expressed by the following Expression (1), and is also referred to as a hidden variable, an intermediate expression, a latent expression, or the like.

$$z = μ + σ ε \qquad ( 1 )$$

$$ε ∼ N ( 0 , 1 ) \qquad ( N : standard normal distribution )$$

The latent variable z output from the VIB 22 is input to the attention module 24.

The query generator 23 generates data to be input to the attention module 24 as a query (hereinafter, the data is referred to as “query data”). For example, the query generator 23 generates query data based on a predetermined initial value (random seed) by a random number generator, and outputs the query data to the attention module 24.

The query data generated by the query generator 23 is input to the attention module 24 as a query, and the latent variable z generated by the VIB 22 is input to the attention module 24 as a key and a value. The attention module 24 applies a weight indicating the similarity between the query and the key to the value, and emphasizes and outputs information having a high degree of relevance in the latent variable z.

FIG. 4C illustrates a configuration example of the attention module 24. The example of FIG. 4C corresponds to the above-described perceiver, and is configured by connecting a cross-attention mechanism and a self-attention mechanism. In other words, the attention module 24 is a transformer obtained by stacking cross-attention and self-attention. The attention module 24 performs information compression to extract highly relevant information extracted by the cross-attention and the self-attention. In this case, it can be considered that the attention module 24 performs information bottleneck processing of limiting the input information by the number of dimensions of the query data, that is, information compression. The attention module 24 outputs the latent variable z, in which highly relevant information is emphasized, to the pooling layer 25. Note that, although one attention module 24 is provided in FIG. 3, multiple attention modules 24 may be configured in multiple layers. Basically, by increasing the number of attention modules 24, the effect of extracting information with a high degree of relevance by the attention mechanism can be expected to increase.

The pooling layer 25 is configured by an average pooling layer, a max pooling layer, and the like, and outputs the latent variable z output from the attention module 24 to the decoder 26 as a vector having a predetermined spatial size.

The decoder 26 calculates and outputs a disease risk score (hereinafter referred to as a “risk score”) Y corresponding to the input data based on the latent variable z output from the pooling layer 25. FIG. 4D illustrates a configuration example of the decoder 26. The decoder 26 can be a classifier that outputs the disease risk score from the input latent variable z.

The optimization unit 29 receives the latent variable z output from the VIB 22 and receives a risk score Y output from the decoder 26. In addition, a ground truth for the input data is prepared in advance as the training data, and the ground truth corresponding to the input data is input to the optimization unit 29. The optimization unit 29 calculates a loss based on the latent variable z, the risk score Y, and the ground truth. The optimization unit 29 can use a loss function expressed by the following Formula (2).

$$L = {L}_{d} ( y , \hat{y} ) + β * K L D \qquad ( 2 )$$

Here, the first term of Formula (2) indicates a difference between the risk score Y (=y\hat{ }), which is an estimated value of the disease risk, and the ground truth value y. In addition, KLD in the second term of Formula (2) indicates Kullback-Leibler (KL) divergence between the latent variable z and a predetermined reference distribution (for example, normal distribution). The optimization unit 29 optimizes the parameters of the neural network NN constituting the encoder 21, the VIB 22, the attention module 24, the pooling layer 25, and the decoder 26 so as to minimize the loss L. Thus, the training of the risk estimation model is performed.

### (Training Processing)

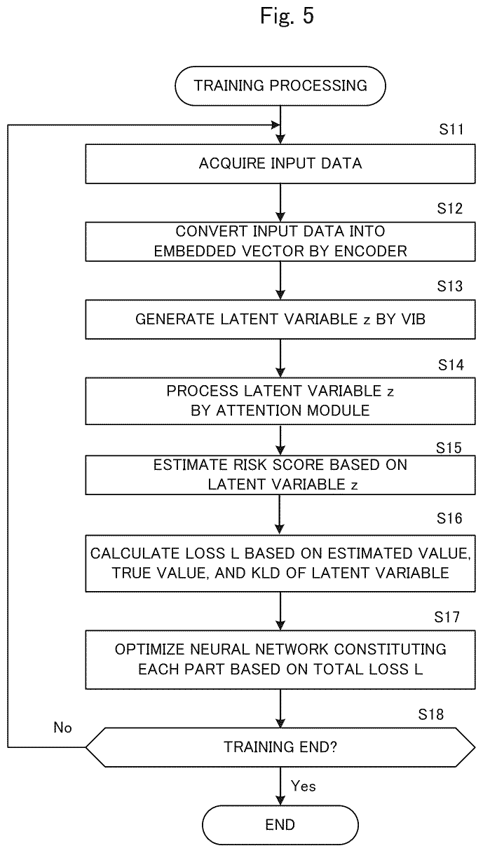

Next, the training processing executed by the above training device 20 will be described. FIG. 5 is a flowchart of the training processing. This processing is achieved by the processor 11 illustrated in FIG. 2 executing a program prepared in advance and operating as the components illustrated in FIG. 3.

First, the encoder 21 acquires input data included in the training data (step S11). Next, the encoder 21 converts the input data into a vector in the latent space (step S12). Next, the VIB 22 calculates a probability distribution of the input vector in the latent space, and generates the latent variable z indicating the probability distribution (step S13). Next, the attention module 24 receives query data from the query generator 23 and performs attention processing on the latent variable z output from the VIB 22 (step S14). The latent variable z subjected to the attention processing is input to the decoder 26 after being subjected to pooling processing. The decoder 26 estimates the risk score based on the input latent variable z (step S15).

Next, the optimization unit 29 calculates the loss L using the latent variable z output from the VIB 22, the risk score Y output from the decoder 26, and the ground truth (step S16), and optimizes the parameters of the neural network NN so as to minimize the loss L (step S17).

Next, the training device 20 determines whether a predetermined training end condition is satisfied (step S18). Examples of the training end condition include that a predetermined number of pieces of attribute data prepared as training data has been used, that the loss L has become equal to or less than a predetermined value, and that the loss L has converged. If the training end condition is not satisfied (step S18: No), the process returns to step S11. On the other hand, if the training end condition is satisfied (step S18: Yes), the training processing ends.

### [Estimation Phase]

Next, the estimation phase by the risk estimation device 100 will be described. In the estimation phase, the risk estimation device 100 estimates the disease risk of a certain subject based on input data on the health of the subject. At this time, the risk estimation device 100 uses the risk estimation model trained in the above training phase, specifically, the trained neural network NN.

### (Risk Estimation Device)

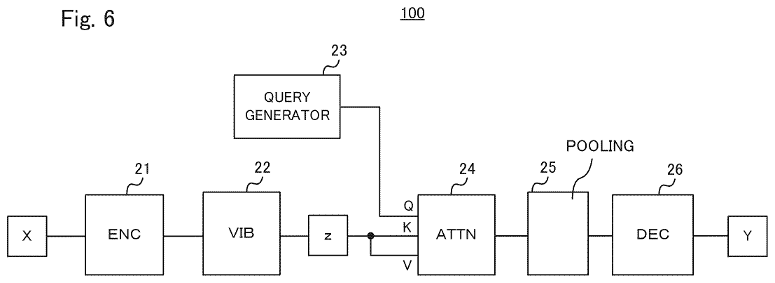

FIG. 6 is a block diagram illustrating a functional configuration of the risk estimation device. The risk estimation device 100 includes the query generator 23 and the neural network NN optimized in the training phase, that is, the encoder 21, the VIB 22, the attention module 24, the pooling layer 25, and the decoder 26.

In the estimation phase, each component basically operates similarly to the training phase with respect to input data of a certain subject. First, the encoder 21 converts the input data into an embedding vector in the latent space. The VIB 22 calculates a probability distribution in the latent space from the vector and generates the latent variable z. The query generator 23 generates query data based on a random number, and the attention module 24 executes attention processing based on the query data and the latent variable z. The latent variable z subjected to the attention processing is pooled and input to the decoder 26. The decoder 26 estimates and outputs the risk score Y of the subject based on the input latent variable z.

In the above configuration, the encoder 21 is an example of a conversion means, the VIB 22 is an example of a first processing means, and the query generator 23 is an example of a query generation means. The attention module 24 is an example of a second processing means, and the decoder 26 is an example of an estimation means.

### (Risk Estimation Processing)

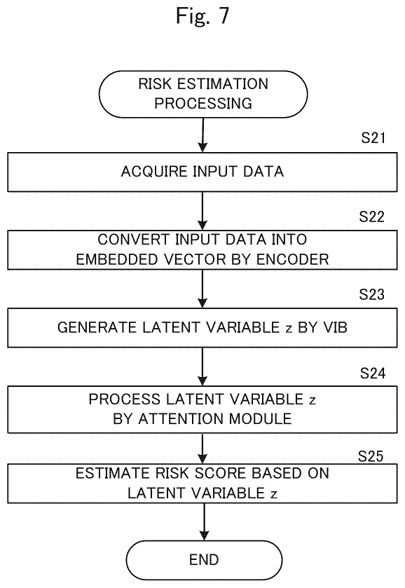

Next, the risk estimation processing executed by the risk estimation device 100 will be described. FIG. 7 is a flowchart of the risk estimation processing. This processing is achieved by the processor 11 illustrated in FIG. 2 executing a program prepared in advance and operating as each element illustrated in FIG. 6.

First, the encoder 21 acquires input data on the health of the subject (step S21). Next, the encoder 21 converts the input data into an embedding vector in the latent space (step S22). Subsequently, the VIB 22 calculates a probability distribution in the latent space based on the embedding vector, and generates the latent variable z (step S23). Next, the query generator 23 generates query data based on a random number, and the attention module 24 executes attention processing based on the query data and the latent variable z (step S24). After the attention processing, the latent variable z is pooled and input to the decoder 26. The decoder 26 estimates and outputs the risk score Y of the subject based on the input latent variable z (step S25). Then, the risk estimation processing ends.

### [Verification of Effect]

### (Verification 1)

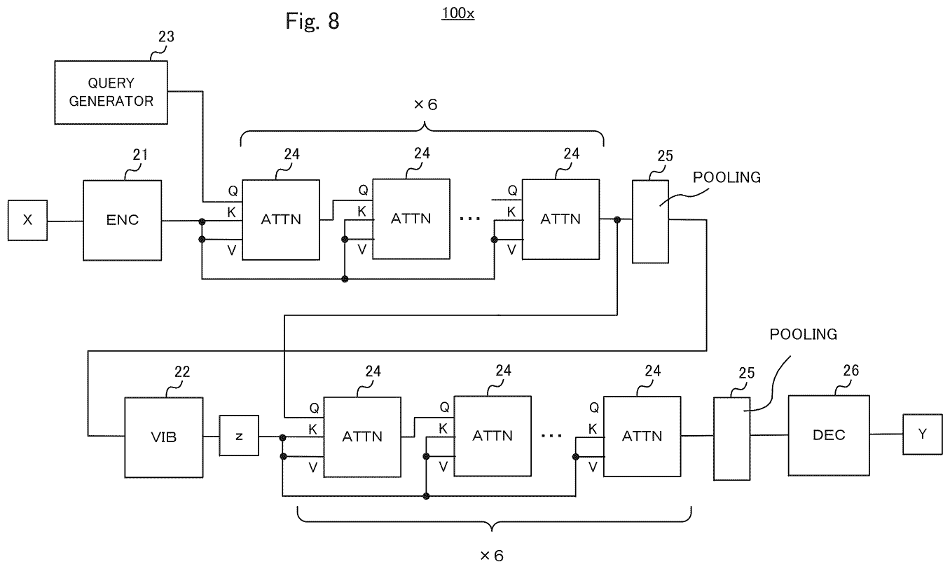

The effects of the risk estimation device 100 according to the example embodiment were verified by experiments. Specifically, using a risk estimation device 100x illustrated in FIG. 8, the risk score of a disease was calculated from input health data. In the risk estimation device 100x, six attention modules 24 were arranged between the encoder 21 and the VIB 22 and between the VIB 22 and the pooling layer 25. As the pooling layer 25, average pooling was used.

As the input data, health data collected from many persons was used. Specifically, sex, age, weight, cholesterol value, blood glucose, smoking history, drinking history, blood pressure, BMI, human body activity, and the like were used as the input data. The collected data was divided into training data, validation data and test date with the ratio of 6:2:2. Random seeds in the query generator 23 were fixed.

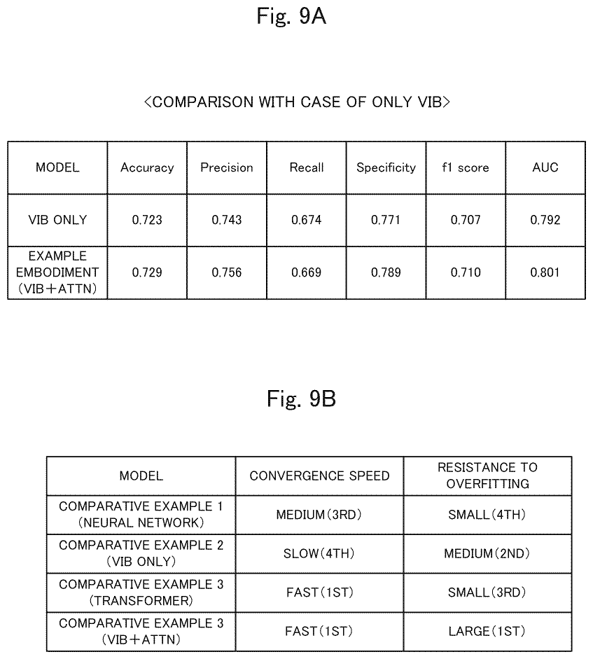

FIG. 9A illustrates a comparison between an estimation result by a configuration using only the VIB for the risk estimation device 100 and an estimation result by the risk estimation device 100x according to the above example embodiment. As indices of comparison between the estimation results, accuracy, precision, recall, specificity, F1 score, and area under the curve (AUC) were used. It is understood that the configuration of the example embodiment using the VIB and the attention module has better performance in either index.

### (Verification 2)

As another verification, in the risk estimation device 100x illustrated in FIG. 8, a risk estimation device 100y in which the number of connected attention modules, which has been six, was reduced to three was used. This is set as the device of the example embodiment. As objects of comparison, the convergence speed in the calculation of the risk score and the influence of overfitting were compared for a risk estimation device configured by a simple neural network (Comparative Example 1), a risk estimation device using only the VIB (Comparative Example 2), a risk estimation device using a transformer including cross-attention and self-attention (corresponding to the above-described perceiver) (Comparative Example 3), and the risk estimation device 100y (example embodiment).

FIG. 9B is a table comparing the convergence speed in the calculation of the risk score and the resistance to overfitting among the above four risk estimation devices. Regarding the convergence speed, Comparative Example 3 and the example embodiment are almost equally fast, Comparative Example 1 is the second fastest, and Comparative Example 2 is the slowest. Meanwhile, regarding the resistance to overfitting, the example embodiment is the highest, Comparative Example 2 is the second highest, Comparative Example 3 is the third highest, and Comparative Example 1 is the lowest. As described above, it can be seen that the risk estimation device of the example embodiment using the VIB and the attention module is superior to the comparative examples in both the convergence speed in the calculation of the risk score and the resistance to overfitting.

### MODIFICATION

Next, modifications of the above example embodiment will be described. The following modifications can be appropriately combined and applied to the above example embodiment.

### First Modification

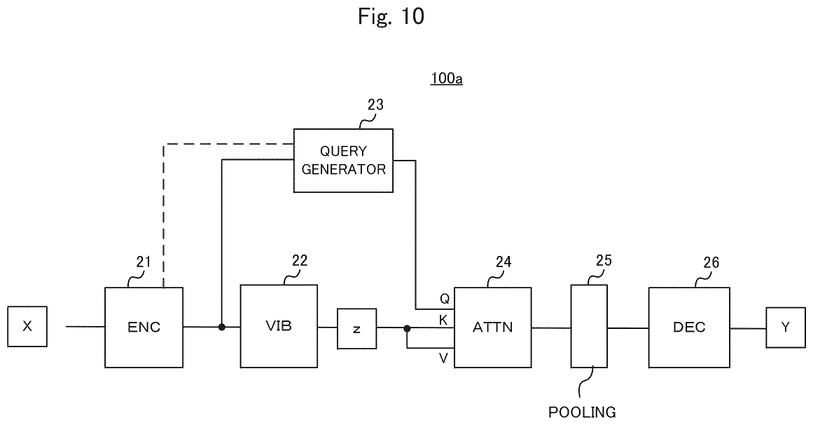

FIG. 10 illustrates one modification of the risk estimation device. A risk estimation device 100a of a first modification illustrated in FIG. 10 is different from the risk estimation device 100 illustrated in FIG. 6 in that an output of the encoder 21 or an output of an intermediate layer of the encoder 21 is input to a query generator 23. In the risk estimation device 100 illustrated in FIG. 6, the query generator 23 generates query data using a random number. On the other hand, the risk estimation device 100a of the first modification generates query data based on the vector output from the encoder 21. This makes the query data highly related to the original input data, so that the risk estimation device 100a can calculate the risk score with higher accuracy.

### Second Modification

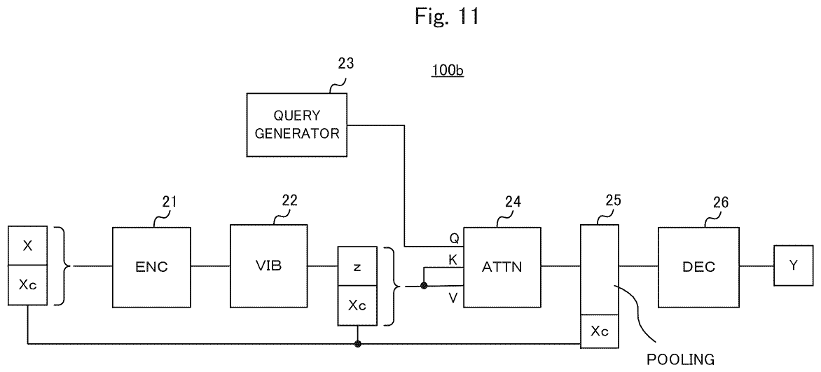

FIG. 11 illustrates another modification of the risk estimation device. A risk estimation device 100b of a second modification illustrated in FIG. 11 is different from the risk estimation device 100 illustrated in FIG. 6 in that a condition variable is used as an input. That is, a condition variable Xc is input to the risk estimation device 100b of the second modification in addition to the input data X corresponding to an explanatory variable. The condition variable Xc is input to the encoder 21, is added to the latent variable z output from the VIB 22, and is input to the attention module 24. Note that, multiple attention modules 24 may be connected in multiple layers. Further, the condition variable Xc is added to the pooling layer 25.

By adding the condition variable Xc, it is possible to estimate the disease risk under a certain condition. For example, by adding the age and sex of the subject as the condition variable Xc, it is possible to estimate the disease risk under the conditions of the age and sex. Note that, the Conditional variational information bottleneck (conditional VIB) is described in Document 3 below, and this document is incorporated herein by reference.
(Document 3) Choi, M., & Lee, C. (2023). Conditional Information Bottleneck Approach for Time Series Imputation. In The Twelfth International Conference on Learning Representations

### Third Modification

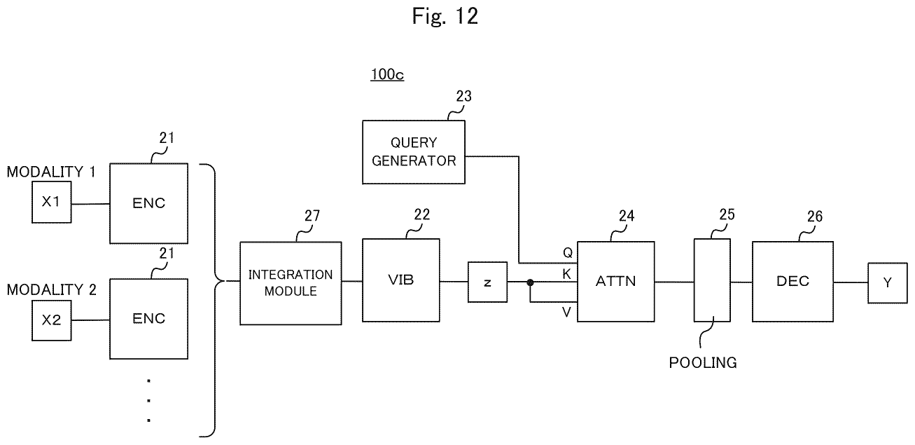

FIG. 12 illustrates another modification of the risk estimation device. A risk estimation device 100c of a third modification illustrated in FIG. 12 is different from the risk estimation device 100 illustrated in FIG. 6 in that data of multiple modalities is used as an input. Specifically, the encoder 21 is provided to each of the multiple modalities, and outputs of the multiple encoders 21 are integrated by an integration module 27 and input to the VIB 22. The integration module 27 may calculate Product of expert of outputs of the multiple encoders 21, may calculate Mixture of expert, or may concatenate outputs of the multiple encoders 21. An attention module may be used as the integration module 27. Note that, multiple attention modules 24 may be connected in multiple layers.

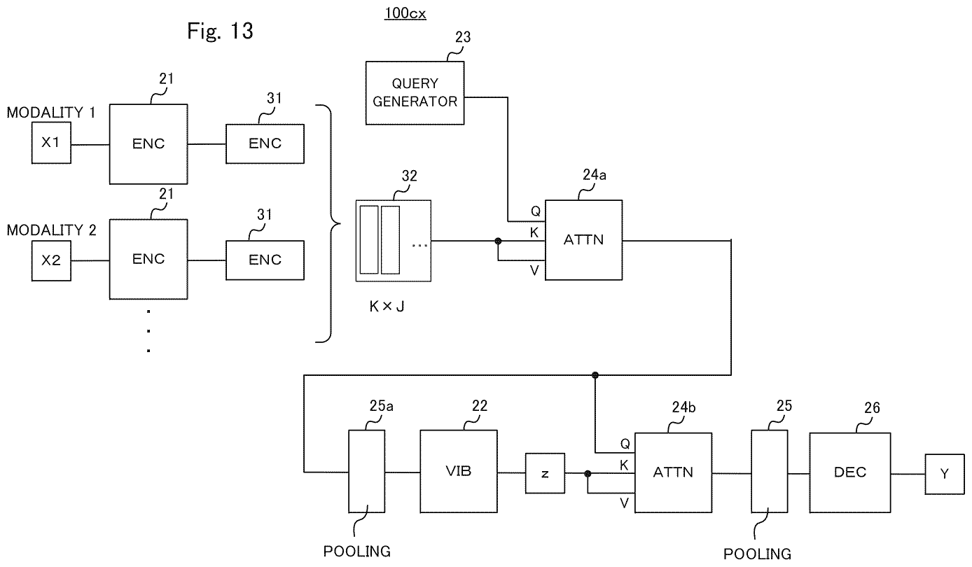

FIG. 13 illustrates a configuration example of the risk estimation device in a case where an attention module is used as an integration module 27. In a risk estimation device 100cx of FIG. 13, in addition to the encoder 21, an encoder 31 is provided for each of multiple modalities. The encoder 31 converts the vector, output from the encoder 21, into a J-dimensional vector. As a result, a K×J array 32 is generated and input to the key and value of an attention module 24a. An attention module 24a performs attention processing using the above-described array 32 and query data from the query generator 23, and outputs the processed data to a pooling layer 25a and an attention module 24b. The subsequent processing is similar to that of the risk estimation device 100c illustrated in FIG. 12. In this example, the attention module 24a generates data obtained by integrating data of the multiple modalities.

### Fourth Modification

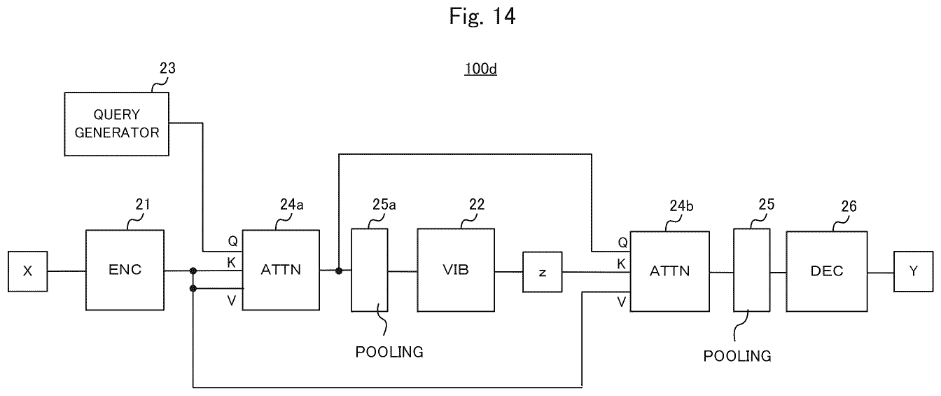

FIG. 14 illustrates another modification of the risk estimation device. A risk estimation device 100d according to a fourth modification illustrated in FIG. 14 is different from the risk estimation device 100 illustrated in FIG. 6 in that an attention module 24a and a pooling layer 25a are provided between the encoder 21 and the VIB 22. The vector output from the encoder 21 is input to the key and value of the attention module 24a and is input to the value of the attention module 24b. To the attention module 24b, the output of the attention module 24a is input as a query, the output of the VIB 22 is input as a key, and the output of the encoder 21 is input as a value. That is, linear conversion data by the encoder 21, nonlinear conversion data by the attention module 24a, and probability distribution conversion data by the VIB 22 are input to the attention module 24b. Accordingly, the attention module 24b can extract the relevance of the three of the linear conversion, the nonlinear conversion, and the probability distribution change and use the extracted relevance for risk estimation. Note that, the attention modules 24a, 24b may be obtained by connecting multiple attention modules 24 in multiple layers.

### Fifth Modification

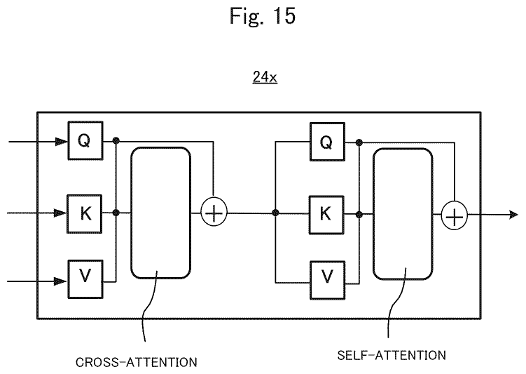

FIG. 15 illustrates a modification of the attention module 24. As can be understood from comparison with FIG. 4C, an attention module 24x according to the fifth modification is different from the attention module 24 of FIG. 4C in that a query is added to the outputs of the cross-attention and the self-attention for normalization. As a result, the portion of the query is emphasized in the attention processing, so that the relevance of the data can be further emphasized, and the convergence of the operation becomes faster. The attention module 24x of the fifth modification can be applied to the above-described example embodiment and the first to fourth modifications.

### Sixth Modification

In the first example embodiment described above, the risk estimation device is applied to generate attribute data on human health, but the application of the present disclosure is not limited thereto. For example, the present disclosure may be applied to inspection and diagnosis of a machine or device. That is, the method of the present disclosure may be applied to estimate the state of the machine or device based on data of multiple modalities detected and collected in inspection or diagnosis.

### Second Example Embodiment

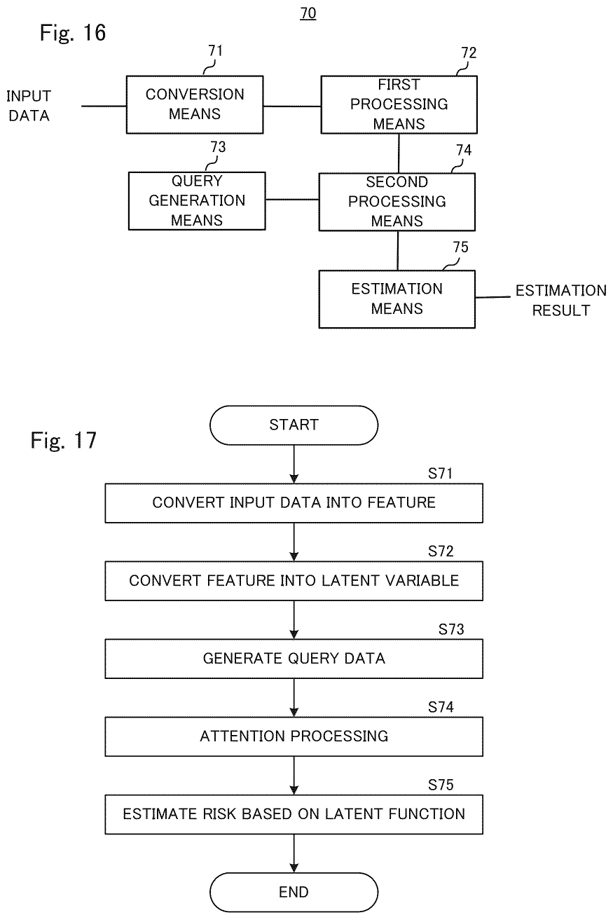

FIG. 16 is a block diagram illustrating a functional configuration of a risk estimation device of a second example embodiment. A risk estimation device 70 includes a conversion means 71, a first processing means 72, a query generation means 73, a second processing means 74, and an estimation means 75.

FIG. 17 is a flowchart of processing by the risk estimation device according to the second example embodiment. The conversion means 71 converts input data into a feature (step S71). The first processing means 72 converts the feature into a latent variable indicating a probability distribution in the latent space (step S72). The query generation means 73 generates query data (step S73). The second processing means 74 performs attention processing on the query data and the latent variable using the attention mechanism, and outputs the latent variable after the attention processing (step S74). The estimation means 75 estimates a risk based on the latent variable after the attention processing (step S75).

According to the risk estimation device 70 of the second example embodiment, it is possible to estimate the risk with high accuracy.

A part or all of the example embodiments described above may also be described as the following supplementary notes, but not limited thereto.

### Supplementary Note 1

A risk estimation device comprising:
a conversion means configured to convert input data into a feature;
a first processing means configured to convert the feature into a latent variable
indicating a probability distribution in a latent space;
a query generation means configured to generate query data;
a second processing means configured to perform attention processing on the query data and the latent variable using an attention mechanism, and output the latent variable after the attention processing; and
an estimation means configured to estimate a risk based on the latent variable after the attention processing.

### Supplementary Note 2

The risk estimation device according to Supplementary note 1, wherein the attention processing includes cross-attention processing and self-attention processing.

### Supplementary Note 3

The risk estimation device according to Supplementary note 1, wherein the query data is input to the attention mechanism as a query, and the latent variable is input to the attention mechanism as a key and a value.

### Supplementary Note 4

The risk estimation device according to Supplementary note 3, wherein the query generation means generates the query data based on the feature output from the conversion means.

### Supplementary Note 5

The risk estimation device according to Supplementary note 1,
wherein the conversion means converts the input data and a condition variable into a feature, and
wherein the second processing means performs the attention processing on the latent variable and the condition variable.

### Supplementary Note 6

The risk estimation device according to Supplementary note 1,
wherein the input data includes data of a plurality of different modalities,
wherein the conversion means converts the data of the plurality of modalities into features of the plurality of modalities,
wherein the risk estimation device further includes an integration means for integrating the features of the plurality of modalities to generate an integrated feature, and
wherein the first processing means converts the integrated feature into the latent variable.

### Supplementary Note 7

The risk estimation device according to Supplementary note 1,
wherein the first processing means, the second processing means, and the estimation means are configured by a deep learning model,
wherein the first processing means performs information compression by a VIB, and
wherein the second processing means performs information compression by the attention mechanism.

### Supplementary Note 8

The risk estimation device according to Supplementary note 1,
wherein the input data is data related to health of a subject, and
wherein the estimation means estimates a disease risk of the subject by a learned machine learning model.

### Supplementary Note 9

A risk estimation method executed by a computer, the risk estimation method comprising:
converting input data into a feature;
converting the feature into a latent variable indicating a probability distribution in a latent space;
generating query data;
performing attention processing on the query data and the latent variable using an attention mechanism and outputting the latent variable subjected to the attention processing; and
estimating a risk based on the latent variable subjected to the attention processing.

### Supplementary Note 10

A program causing a computer to execute processing comprising:
converting input data into a feature;
converting the feature into a latent variable indicating a probability distribution in a latent space;
generating query data;
performing attention processing on the query data and the latent variable using an attention mechanism and outputting the latent variable subjected to the attention processing; and
estimating a risk based on the latent variable subjected to the attention processing.

Some or all of the configurations described in Supplementary Notes 2 to 8 dependent on the above-described Supplementary Note 1 can also be dependent on Supplementary Notes 9 and 10 by a dependency relationship similar to that of Supplementary Notes 2 to 8. Some or all of the configurations described as the Supplementary Notes can be similarly dependent on not only the Supplementary Notes 1, 9, and 10, but also diverse pieces of hardware and software, various recording means for recording software, or systems without departing from the above-described example embodiments.

While the present disclosure has been particularly shown and described with reference to example embodiments and examples thereof, the present disclosure is not limited to these example embodiments and examples. It will be understood by those of ordinary skill in the art that various changes in form and details may be made therein without departing from the spirit and scope of the present disclosure as defined by the claims.

### DESCRIPTION OF SYMBOLS

11 Processor
20 Training device
21 Encoder
22 VIB
23 Query generator
24, 24a, 24b Attention module
25 Pooling layer
16 Decoder
29 Optimization unit
100, 100a, 100b, 100c, 100cx, 100d Risk estimation device

1. A risk estimation device comprising:
a memory configured to store instructions; and
an interface configured to receive, as the input data, health data of a subject obtained in a regular medical examination; and
a processor configured to execute the instructions to:
convert the input data into an embedding vector that is a feature in a latent space, wherein the converting is performed by an encoder including a dropout layer;
calculate a probability distribution in the latent space of the embedding vector, the probability distribution being indicated by a mean μ and a standard deviation σ, and generate a latent variable indicating the probability distribution;
generate query data based on (i) a predetermined initial value by a random number generator, or (ii) an output of the encoder or an output of an intermediate layer of the encoder;
perform attention processing on the query data and the latent variable using an attention mechanism, and output the latent variable after the attention processing, wherein
the attention processing applies a weight indicating a similarity between a query and a key to a value to emphasize information having a high degree of relevance in the latent variable,
the attention mechanism performs information bottleneck processing by limiting input information based on a number of dimensions of the query data, and
the attention processing adds the query to an output of the attention processing for normalization;
pool the latent variable after the attention processing by a pooling layer to obtain a pooled latent variable; and
estimate a disease risk score of the subject based on the pooled latent variable, and output an estimation result of the disease risk score to a display device or another external device.

2. The risk estimation device according to claim 1, wherein the attention processing includes cross-attention processing and self-attention processing.

3. The risk estimation device according to claim 1, wherein the query data is input to the attention mechanism as a query, and the latent variable is input to the attention mechanism as a key and a value.

4. The risk estimation device according to claim 3, wherein the processor generates the query data based on the feature output from the encoder.

5. The risk estimation device according to claim 1,
wherein the processor converts the input data and a condition variable into a feature, and
wherein the processor performs the attention processing on the latent variable and the condition variable.

6. The risk estimation device according to claim 1,
wherein the input data includes data of a plurality of different modalities,
wherein the processor converts the data of the plurality of modalities into features of the plurality of modalities,
wherein the processor is further configured to execute the instructions to integrate the features of the plurality of modalities to generate an integrated feature, and
wherein the processor converts the integrated feature into the latent variable.

7. The risk estimation device according to claim 1,
wherein the processor includes a deep learning model,
wherein the processor performs information compression by a VIB, and
wherein the processor performs information compression by the attention mechanism.

8. The risk estimation device according to claim 1,
wherein the input data is data related to health of a subject, and
wherein the processor estimates a disease risk of the subject by a learned machine learning model.

9. A risk estimation method executed by a computer, the risk estimation method comprising:
receiving, as input data, health data of a subject obtained in a regular medical examination;
converting the input data into an embedding vector that is a feature in a latent space, wherein the converting is performed by an encoder including a dropout layer;
calculating a probability distribution in the latent space of the embedding vector, the probability distribution being indicated by a mean μ and a standard deviation σ, and generating a latent variable indicating the probability distribution;
generating query data based on (i) a predetermined initial value by a random number generator, or (ii) an output of the encoder or an output of an intermediate layer of the encoder;
performing attention processing on the query data and the latent variable using an attention mechanism and outputting the latent variable after the attention processing, wherein
the attention processing applies a weight indicating a similarity between a query and a key to a value to emphasize information having a high degree of relevance in the latent variable,
the attention mechanism performs information bottleneck processing by limiting input information based on a number of dimensions of the query data, and
the attention processing adds the query to an output of the attention processing for normalization;
pooling the latent variable after the attention processing by a pooling layer to obtain a pooled latent variable; and
estimating a disease risk score of the subject based on the pooled latent variable, and outputting an estimation result of the disease risk score to a display device or another external device.

10. A non-transitory computer-readable recording medium storing instructions that, when executed by a computer, cause the computer to perform processing comprising:
receiving, as input data, health data of a subject obtained in a regular medical examination;
converting the input data into an embedding vector that is a feature in a latent space, wherein the converting is performed by an encoder including a dropout layer;
calculating a probability distribution in the latent space of the embedding vector, the probability distribution being indicated by a mean μ and a standard deviation σ, and generating a latent variable indicating the probability distribution;
generating query data based on (i) a predetermined initial value by a random number generator, or (ii) an output of the encoder or an output of an intermediate layer of the encoder;
performing attention processing on the query data and the latent variable using an attention mechanism, and outputting the latent variable after the attention processing, wherein
the attention processing applies a weight indicating a similarity between a query and a key to a value to emphasize information having a high degree of relevance in the latent variable,
the attention mechanism performs information bottleneck processing by limiting input information based on a number of dimensions of the query data, and
the attention processing adds the query to an output of the attention processing for normalization;
pooling the latent variable after the attention processing by a pooling layer to obtain a pooled latent variable; and
estimating a disease risk score of the subject based on the pooled latent variable, and outputting an estimation result of the disease risk score to a display device or another external device.
